(19) United States
(12) Patent Application Publication
(10) Pub. No.: US 2025/0177002 A1
(43) Pub. Date: Jun. 5, 2025

(54) BIRTHING DEVICE

(71) Applicant: BIRTHGLIDE LIMITED, Alton Hampshire (GB)

(72) Inventor: Tianze GUO, Alton Hampshire (GB)

(73) Assignee: BIRTHGLIDE LIMITED, Alton Hampshire (GB)

(21) Appl. No.: 18/833,688

(22) PCT Filed: Feb. 1, 2023
(86) PCT No.: PCT/GB2023/050223

§ 371 Date: Jul. 26, 2024

(30) Foreign Application Priority Data
Feb. 1, 2022 (GB) 2201260.3
Sep. 22, 2022 (GB) 2213813.5

(51) Int. Cl. A61B 17/42; A61B 17/00; A61F 13/12; A61L 31/04; A61L 31/06; A61L 31/14

(52) U.S. Cl. CPC A61B 17/42; A61F 13/12; A61L 31/048; A61L 31/06; A61L 31/145; A61B 2017/00849; A61B 2017/00858; A61L 2300/452

(57) ABSTRACT

The present invention relates to a device for assisting childbirth and/or preventing obstructed labour, a method of making the device, and a method of assisting childbirth and/or preventing obstructed labour. The invention also extends to use of a hydrogel to assist childbirth and/or prevent obstructed labour.

### TECHNICAL FIELD

The present disclosure relates to a device for assisting childbirth. The disclosure also relates to a method of making the device, a method of assisting childbirth using the device, and use of a hydrogel to assist childbirth.

### BACKGROUND

Labour can be divided into three stages: Stage 1: From the diagnosis of labour to full dilation of the cervix (10 cm); Stage 2: From the full dilation of the cervix to the delivery of the foetus. This usually lasts less than 2 hours in nulliparous women and 1 hour in multiparous women; and Stage 3: From the delivery of the baby until complete delivery of the placenta and the membranes.

During the second stage of labour, the foetal head experiences significant friction from the mother's birth canal. The performance of a natural lubricant, which comprises a mixture of amniotic fluid, vernix caseosa and vaginal fluid, varies from person to person. The natural lubricant may not fully cover the sliding surface throughout the delivery process. Contact between the exposed skin and the birth canal in the absence of the natural lubricant can significantly increase the frictional force generated during labour and thus cause damage to the fragile mucus membrane of the birth canal. Damage to the mucus membrane induces local swelling, which further increases the frictional force generated during labour.

An artificial lubricant, such as a water-based lubricating gel, may be introduced into the birth canal during birth in order to alleviate this problem. However, the driving force provided by uterine contractions and maternal pushing comes in waves. Consequently, the motion of the foetus through the birth canal follows a start-stop pattern at slow speed. Under such conditions, liquid lubricant may be pushed away from the sliding surface, exposing the sliding surface to boundary lubrication, which results in a significant increase in friction. In addition, water-based gel lubricants may reduce friction when initially applied. However, after a short duration, friction usually increases significantly due to the loss of water content because of factors such as evaporation and/or absorption. Increased friction can make the delivery of a baby even more difficult, particularly during obstructed labour.

Obstructed labour occurs (during stage 2) when despite strong uterine contractions, the presenting part of the foetus cannot progress through the birth canal. If not resolved quickly, it can lead to foetal and maternal complications. Obstructed labour can only be alleviated by means of an operative delivery, either by a caesarean section or by delivery with the aid of instruments (e.g., forceps, vacuum extraction or symphysiotomy).

There is therefore a need for an improved means of assisting childbirth and/or preventing obstructed labour.

### SUMMARY

According to a first aspect of the disclosure, there is provided a device for assisting childbirth, the device comprising:
a hydrogel membrane for covering at least part of a baby in a birth canal during childbirth and/or for lining at least part of a birth canal during childbirth.

A hydrogel membrane for covering at least a part of a baby during childbirth and/or for lining at least part of the birth canal during childbirth may refer to one or more hydrogel membranes that have a total surface area of at least about 1 cm². The one or more hydrogel membranes may have a total surface area of about 1 cm² to about 3,000 cm².

The membrane may be a 2D shape. The membrane may be for wrapping around part of a baby while in a birth canal. For example, the membrane may be a 2D shape that is capable of being wrapped around part of a baby and/or capable of lining part of a birth canal. The membrane may be a (3D) closed loop that can be wrapped around part of a baby while in a birth canal and/or line part of a birth canal. Wrapping may refer to wrapping a baby around its longitudinal axis (circumferentially wrapping) and/or wrapping a baby along its longitudinal axis. Thus, the device (e.g., the hydrogel membrane) may be a sleeve. The hydrogel membrane may be for circumferentially wrapping a baby.

The part of the baby may be the head of the baby, the neck of the baby, the torso of the baby, the limbs of the baby, the arms of the baby or the legs of the baby. At least part of the birth canal may be 10%, 20%, 30%, 40%, 50%, 60%, 70%, 75%, 80%, 85%, 90%, 95%, 96%, 97%, 98%, 99% or 100% of the surface area of the birth canal. At least part of the baby may be at least about 10%, 20%, 30%, 40%, 50%, 60%, 70%, 75%, 80%, 85%, 90%, 95%, 96%, 97%, 98%, 99% or 100% of the surface area of the baby. At least part of the baby may be at least about 10%, 20%, 30%, 40%, 50%, 60%, 70%, 75%, 80%, 85%, 90%, 95%, 96%, 97%, 98%, 99% or 100% of the surface area of the baby's entire body, the head of the baby, the neck of the baby, the torso of the baby, the limbs of the baby, the arms of the baby or the legs of the baby. The device may cover from one part of the baby to another part selected from the group consisting of: the head of the baby, the neck of the baby, the torso of the baby, the limbs of the baby, the arms of the baby, and the legs of the baby.

The hydrogel membrane may comprise one or more (intermittent) non-hydrogel regions, such as strips. The non-hydrogel regions may comprise or be made from a high friction material.

In one embodiment, the hydrogel membrane comprises or is in the form of a crown for being worn on a baby's head and the crown comprises an opening for receiving the baby's head.

Thus, according to another aspect of the disclosure, there is provided a device for assisting childbirth, the device comprising:
a crown for being worn on a baby's head,
wherein the crown comprises an opening for receiving the baby's head, and
wherein the crown is made from or comprises a hydrogel.

The device (e.g., the hydrogel membrane) may comprise a bulbous rim. The entire circumference or periphery of the hydrogel membrane may be or comprise a bulbous rim. Thus, in one embodiment, a bulbous rim defines the circumference or periphery of the hydrogel membrane. In another embodiment, the bulbous rim defines an opening in the crown. Thus, the opening may comprise a bulbous rim.

According to another aspect of the disclosure, there is provided a device for assisting childbirth, the device comprising:
a crown for being worn on a baby's head; and
a bulbous rim defining an opening in the crown;
   wherein the opening is for receiving the baby's head, and
   wherein the crown and the rim are made from or comprise a hydrogel.

The bulbous rim may be made from or comprise a hydrogel. The bulbous rim may be made from or comprise a same hydrogel as the crown or the hydrogel membrane. The bulbous rim may be made from or comprise a non-hydrogel material, e.g., a high friction material. The bulbous rim may comprise one or more (intermittent) non-hydrogel regions, such as strips. The bulbous rim or the non-hydrogel regions may be made from or comprise a high friction material. The high friction material referred to herein may be one or more selected from the group comprising or consisting of rubber, silicone, polyethylene, polyurethane, nylon, polydimethylsiloxane, polyvinylchloride, polyethersulfone, polytetrafluoroethylene, polyetherimide, polycarbonate, polysulfone, polyetheretherketone and polypropylene.

In one embodiment, the device may be a cap. In another embodiment, the device may be a sleeve.

The device according to the invention can be used to assist childbirth and/or prevent obstructed labour. This is achieved by (i) preventing direct (adhesive) contact between the skin of the unborn baby and the mucosal tissue of the birth canal, and (ii) creating a (lubricious) hydrogel surface that contacts the mucosal tissue of the birth canal. In other words, the device acts as an interface between the skin of the unborn baby and the mucosal tissue of the birth canal during childbirth. Furthermore, the self-lubricating property of the hydrogel ensures that the device is consistently lubricated when it is in use. It also enables the device to be used for prolonged periods without becoming dry. Consequently, when the device is in place (in the birth canal), it aids childbirth by:
1) providing a lower degree of friction at the interface of the baby and the device compared to the degree of friction at the interface of the device and the birth canal, thus resulting in the delivery of the baby without the device (this is most likely to occur when the device is in the form of a sleeve and anchored to the birth canal);
2) providing a lower degree of friction at the interface of the device and the birth canal compared to the degree of friction at the interface of the baby and the device, thus resulting in the simultaneous delivery of the baby and the device (this is most likely to occur when the device is a cap worn on the head of the baby);
3) providing a combination of (1) and (2) (this is most likely to occur when the device is oversized); or
4) in embodiments where the device provides a plurality of hydrogel surfaces between the baby and the mucosal tissue of the birth canal, providing a lower degree of friction at the interface of the hydrogel surfaces so that the surfaces slide with respect to each other (and thus encouraging delivery of the baby), or providing a combination of (1) to (3).

The hydrogel of the device may be reinforced with synthetic polymer fibres. Therefore, the hydrogel membrane, the hydrogel of the crown, or the hydrogel of the sleeve May be reinforced with synthetic polymer fibres. The hydrogel of the bulbous rim may be reinforced with synthetic polymer fibres. The hydrogel of the crown or the hydrogel membrane, or the hydrogel of the sleeve may be reinforced with synthetic polymer fibres and the hydrogel of the bulbous rim may not be reinforced with synthetic polymer fibres. The hydrogel of the bulbous rim may be reinforced with synthetic polymer fibres and the hydrogel of the crown, the membrane or the hydrogel of the sleeve may not be reinforced with synthetic polymer fibres. The hydrogel of the bulbous rim may be reinforced with synthetic polymer fibres and the hydrogel of the crown, the hydrogel of the membrane or the hydrogel of the sleeve may not be reinforced.

An embodiment of the device (e.g., the sleeve) may also be for covering the torso of a baby. Thus, the device or crown may be for covering from a baby's head through to its torso.

An embodiment of the device or crown may be a cap. In this embodiment, the opening in the crown is for receiving the circumference of a baby's head. Thus, the cap may be for covering from the top (or crown) of a baby's head through to the circumference of a baby's head or through to the neck of the baby. The cap may be at least worn on a baby's head. The cap may be for covering the torso of a baby. Thus, the cap may be for covering from the top of a baby's head through to the torso of the baby. The cap May be for covering from the top of a baby's head through to and including the feet of the baby. The crown may be folded to create a double layer of hydrogel between the baby's head and the mucosal tissue of the birth canal.

An embodiment of the device or a crown may be a sleeve. The sleeve may be used to assist childbirth by creating a lubricious surface that prevents direct contact between the skin of the baby (e.g., the head only, or part of or the entire body of the baby) and the mucosal tissue of the birth canal. The sleeve may comprise a first opening and a second opening at the opposite end. The sleeve may have a first bulbous rim defining a first opening at one end and a second opening at the other end. The sleeve may further comprise a (third) bulbous rim located between the first bulbous rim and the second opening. The second opening may be defined by a second bulbous rim. Therefore, the sleeve may comprise a second bulbous rim located at the second opening. The first bulbous rim may be for sitting on top of a baby's head. Thus, the first bulbous rim or the first opening may have a diameter (or largest length) smaller than that of a baby's head. The second and/or third bulbous rim or the second opening may have a diameter (or largest length) greater than that of the first bulbous rim. The second and/or third bulbous rim or the second opening may have a diameter (or largest length) greater than that of a baby's head. The second and third bulbous rim may be the same length in circumference or have the same diameter. The sleeve may be used as a crown. Thus, the crown or sleeve may be for at least being worn on a baby's head. The sleeve may be for covering the torso of a baby. Thus, the sleeve may be for covering from the baby's head through to the torso of the baby. The sleeve may be for covering from the baby's head through to and including the feet of the baby. The sleeve may be folded to create a double layer of sleeve material between the baby's head (and optionally the baby's body) and the birth canal surface. The double layer of hydrogel enables easier passage of the baby through the birth canal.

The crown may be any shape that can be used to at least cover or be worn on the head of a baby. Thus, in one embodiment, the cap or the sleeve may be the shape of a cone.

The head of the baby is usually the most challenging body part to push out of the birth canal because of its relative size. Once the head has fully emerged from the birth canal, the rest of the body can be pushed out with relative ease. Thus, the cap may only cover the head of a baby. The cap may cover at least about 50%, at least about 60%, at least about 70%, at least about 80%, or at least about 90% of a baby's head while it is in the birth canal. The sleeve may, however, be used to cover at least about 50%, at least about 60%, at least about 70%, at least about 80%, or at least about 90% of a baby's body while it is in the birth canal.

The crown may be made from or comprise one or more separate panels of hydrogel. The crown of the cap may comprise a handle to aid removal of the cap. The cap may comprise a handle connected to the crown via an extraction cord in order to aid removal of the cap. The extraction cord may be attached to the upper half and outer surface of the crown. The crown may be dome shaped.

The opening of the cap may have a diameter (or largest length) of more than 7 cm, or more than 8 cm, or more than 9 cm. Therefore, the opening of the cap may have a circumference of more than 22 cm, or more than 25 cm, or more than 28 cm. The opening of the cap may have a diameter between about 7 cm and about 15 cm, between about 7 cm and about 12 cm, preferably between about 8 cm and about 14 cm. The opening of the cap may have a circumference between about 22 cm and about 47 cm, between about 22 cm and about 37 cm, between about 25 cm and about 44 cm, or between about 26 cm and about 40 cm. Preferably, the circumference is at least about 24 cm, or about 24 cm to about 40 cm, or about 40 cm.

The first opening of the sleeve may have a diameter (or largest length) of less than 12 cm, or less than 11 cm, or less than 10 cm, or less than 9 cm, or less than 8 cm. Therefore, the first opening of the sleeve may have a circumference of less than 37 cm, or less than 35 cm, or less than 31 cm, or less than 28 cm, or less than 26 cm, or less than 25 cm. The second opening of the sleeve may have a diameter of more than 7 cm, or more than 8 cm, or more than 9 cm, or more than 13 cm. The second opening of the sleeve may have a circumference of more than 22 cm, or more than 25 cm, or more than 28 cm, or more than 40 cm. The first opening of the sleeve may have a diameter (or largest length) of between about 7 cm and about 12 cm. The first opening of the sleeve may have a circumference of between about 22 cm and about 38 cm. The diameter (or largest length) or circumference of the first opening may be smaller than that of the second opening. Thus, the diameter (or largest length) of the second opening may be about 12 cm or greater, about 14 cm or greater, or about 16 cm or greater. Thus, the circumference of the second opening may be about 38 cm or greater, about 40 cm or greater, about 44 cm or greater, about 47 cm or greater, or about 50 cm or greater. The diameter (or largest length) of the second opening may be about 16 cm. The circumference of the second opening may be about 47 cm or about 50 cm.

The skilled person will appreciate that the dimensions of the opening(s) of the device are ultimately determined by how the device is intended to be used. The dimensions of the opening(s) will be determined by the circumference of a new-born baby's head. The circumference of the head of a new-born baby is between 26 cm and 40 cm. However, most new-born babies have a head with a circumference between 30 cm and 37 cm in length. In view of this, in one embodiment, the sleeve may be used to aid delivery of a baby without the sleeve simultaneously exiting the birth canal. Thus, the circumference of the first opening may be greater than about 24 cm and the circumference of the second opening may be greater than about 24 cm, the circumference of the first opening may be greater than about 26 cm and the circumference of the second opening may be greater than about 26 cm, the circumference of the first opening may be greater than about 28 cm and the circumference of the second opening may be greater than about 28 cm, the circumference of the first opening may be greater than about 30 cm and the circumference of the second opening may be greater than about 30 cm, or the circumference of the first opening may be greater than about 32 cm and the circumference of the second opening may be greater than about 32 cm. The circumference of the first opening may be between about 24 cm and 40 cm and the circumference of the second opening may be between about 24 cm and 40 cm, the circumference of the first opening may be between about 26 cm and 40 cm and the circumference of the second opening may be between about 26 cm and 40 cm, the circumference of the first opening may be between about 28 cm and 40 cm and the circumference of the second opening may be between about 28 cm and 40 cm, or the circumference of the first opening may be between about 30 cm and 40 cm and the circumference of the second opening may be between about 30 cm and 40 cm.

In another embodiment, the device (e.g., the sleeve or the cap) may be used to aid delivery of a baby preferably by simultaneously exiting the birth canal with the baby. Thus, the circumference of the first opening of the sleeve may be less than about 30 cm and the circumference of the second opening may be greater than about 37 cm, the circumference of the first opening may be less than about 28 cm and the circumference of the second opening may be greater than about 35 cm, the circumference of the first opening may be less than about 28 cm and the circumference of the second opening may be greater than about 32 cm, the circumference of the first opening may be less than about 26 cm and the circumference of the second opening may be greater than about 30 cm, or the circumference of the first opening may be less than about 26 cm and the circumference of the second opening may be greater than about 28 cm. Preferably, the circumference of the first opening is less than about 26 cm and the circumference of the second opening is greater than about 28 cm. The circumference of the first opening may be between about 0 cm and 30 cm and the circumference of the second opening may be between about 24 cm and 40 cm, the circumference of the first opening may be between about 10 cm and 28 cm and the circumference of the second opening may be between about 26 cm and 40 cm, the circumference of the first opening may be between about 15 cm and 26 cm and the circumference of the second opening may be between about 28 cm and 40 cm, or the circumference of the first opening may be between about 20 cm and 26 cm and the circumference of the second opening may be between about 30 cm and 40 cm.

The circumference of the (first) opening of the cap may be less than about 30 cm, less than about 28 cm, or less than about 26 cm. Preferably the circumference is less than about 26 cm.

The (first and/or second) bulbous rim may be any closed loop shape such as square, oval or circular. The bulbous rim(s) may be oval or circular in shape. Preferably, the bulbous rim(s) form(s) an oval or circular shape along the circumference of the opening. The cross-section of the bulbous rim(s) may be any shape, including square, oval or circular. The cross-section of the bulbous rim(s) may be oval or circular. The thickness of the bulbous rim(s) may be less than about 10 mm, less than about 9 mm, less than about 8 mm, less than about 7 mm, less than about 6 mm, less than about 5 mm, less than about 4 mm, or less than about 3 mm. The thickness of the rim(s) may be greater than about 2 mm, greater than about 3 mm or greater than about 4 mm. The thickness of the rim(s) may be between about 1 mm and 20 mm or about 2 mm and 10 mm. Preferably the thickness of the rim(s) is/are between about 2 mm and about 5 mm. The lubricious nature of the hydrogel makes the crown difficult to grip or handle. The bulbous rim(s) improve(s) the ease with which the device (e.g., the cap, the membrane or the sleeve) can be handled. To further improve the ease with which an embodiment of the device can be handled, the bulbous rim may be made from or comprise a non-hydrogel material, e.g., a high friction material. A high friction material may be one or more selected from the group comprising or consisting of rubber, silicone, polyethylene, polyurethane, nylon, polydimethylsiloxane, polyvinylchloride, polyethersulfone, polytetrafluoroethylene, polyetherimide, polycarbonate, polysulfone, polyetheretherketone and polypropylene.

Part of the bulbous rim may comprise or be made from a hydrogel. Part of the bulbous rim may comprise or be made from a non-hydrogel material. Thus, the bulbous rim may comprise or be made from one or more regions of hydrogel and comprise or be made from one or more regions of a non-hydrogel material (e.g., a high friction material, such as rubber).

The apparatus may comprise a means to aid insertion, retention and/or removal of the device from a birth canal. The means to aid insertion, retention and/or removal of the device from a birth canal may be referred to as a guide. The means to aid insertion, retention and/or removal of the device may be a bulbous rim as referred to herein. The means to aid insertion, retention and/or removal of the device may be a pocket within the membrane, the rim and/or the crown, such that one or more fingers may be inserted therein. The means to aid insertion, retention and/or removal of the device may be a handle or an extraction cord that is attached to or integral with the membrane, the rim and/or the crown. The extraction cord may be secured to the synthetic polymer fibres. The extraction cord may be made from the same material as the synthetic polymer fibres.

The extraction cord may be between about 7 cm and 30 cm in length. Thus, a method of assisting childbirth using an embodiment of the device may comprise pulling the extraction cord. The extraction cord may be situated outside the birth canal during use of the device.

The hydrogel may be made from a 3D network of hydrophilic polymers immersed in a liquid. The liquid may be absorbed by the 3D network of hydrophilic polymers and thus cause the 3D network to swell. Thus, the term “hydrogel” can refer to a 3D network of crosslinked hydrophilic polymers that comprises a liquid, which causes the network to swell. A benefit of using a hydrogel as part of an embodiment is its self-lubricating property. When placed under pressure, hydrogels release the liquid (e.g., water) at the contact surface, thus maintaining fluid-film lubrication and drastically reducing the level of friction (the friction coefficient). In addition, hydrogels are highly absorbent.

The device may be unitary. The device may be a unitary hydrogel or hydrogel membrane. The device may be moulded. The hydrogel does not dissolve in a liquid, such as water. In one embodiment, the hydrogel does not dissolve in a liquid, for example, at a temperature below 45 degrees Celsius. In another embodiment, the hydrogel dissolves in a liquid, for example, at a temperature below 45 degrees Celsius. The hydrogel may be a physically crosslinked hydrogel or a chemically crosslinked hydrogel.

The hydrophilic polymer of the hydrogel may be natural or synthetic. Natural hydrophilic polymers include hyaluronic acid, chitosan, alginate, collagen, silk and fibroin. Synthetic hydrophilic polymers include polyvinyl alcohol (PVA), polyacrylamide (PAAm), a polyhydroxyethylmethacrylate (HEMA) copolymer, polyethylene glycol (PEG), and polydimethylsiloxane (PDMS). The hydrophilic polymer of the hydrogel may be synthetic. The hydrogel may be natural or synthetic.

The hydrogel or the hydrogel membrane may be biocompatible. Thus, the hydrophilic polymer and the fluid may be biocompatible. Biocompatible hydrophilic polymers include PVA, PEG, PAAm, HEMA, PDMS, and mixtures thereof. Thus, the hydrogel or hydrogel membrane may be made from one or more of the polymers selected from the group consisting of: PVA, PEG, PAAm, a HEMA copolymer, and PDMS. The hydrogel or hydrogel membrane may be made from PAAm. Thus, the polymer of the hydrogel may be PAAm. PAAm creates hydrogels with an extremely low friction coefficient.

The hydrogel may be made from a HEMA copolymer such as poly(2-hydroxyethyl methacrylate/methacrylic acid (HEMA-MAA) or HEMA-Vinyl Pyrrolidone (HEMA-VP). Thus, the polymer of the hydrogel may be HEMA-MAA or HEMA-VP. HEMA copolymers create hydrogels that are strong and stretchy, and thus more capable of withstanding tearing forces that are encountered during labour.

The hydrogel may be made from PVA. Thus, the polymer of the hydrogel may be PVA. PVA creates hydrogels that are stretchy, and thus more capable of withstanding tearing forces encountered during labour.

Preferably the hydrophilic polymer is PAAm, PVA, or a HEMA copolymer. Thus, the hydrogel may be made from PAAm, PVA, or a HEMA copolymer. More preferably the hydrophilic polymer is a HEMA copolymer, or PVA. Thus, the hydrogel may be made from PVA, or a HEMA copolymer. Most preferably the hydrophilic polymer is PVA. Thus, the hydrogel may be made from PVA.

The polymer of the hydrogel may be about 5% PVA to about 20% PVA (w/w). A hydrogel made from about 5% PVA to about 20% PVA (w/w) may have a coefficient of friction of about 0.4 or less. Hydrogels made from polymers comprising about 5% PVA have a low coefficient of friction. Preferably the polymer of the hydrogel is about 15% PVA (w/w). Hydrogels made from a polymer comprising about 15% PVA have a low friction coefficient (e.g., about 0.4 or less) and exhibit some elasticity. The polymer of the hydrogel (e.g., HEMA copolymer, PVA or PAAm) may comprise or be mixed with water. Thus, for example, the polymer of the hydrogel may be about 95% water to about 80% water (w/w) and about 5% PVA to about 20% PVA (w/w).

The average molecular weight of the PVA may be between about 47 000 g/mol and about 200 500 g/mol, between about 61 000 g/mol and about 195 000 g/mol, between about 125 000 g/mol and about 195 000 g/mol, between about 125 000 g/mol and about 195 000 g/mol, or between about 145 000 g/mol and about 186 000 g/mol. Preferably the average molecular weight of the PVA is between about 145 000 g/mol and about 186 000 g/mol, more preferably the average molecular weight of the PVA is about 145 000 g/mol. Thus, for example, the polymer of the hydrogel may be about 95% water to about 80% water (w/w) and about 5% PVA to about 20% PVA (w/w) with an average molecular weight of about 145 000 g/mol and about 186 000 g/mol.

The PVA of the hydrogel may be at least about 88% hydrolysed, at least about 92% hydrolysed, at least about 98% hydrolysed, or at least about 99% hydrolysed, or may be 100% hydrolysed. Preferably the PVA is 100% hydrolysed.

A hydrogel made from PVA may have viscosity of about 5 mPa-s to about 60 mPa-s, about 10 mPa-s to about 50 mPa-s, about 15 mPa-s to about 50 mPa-s, or about 20 mPa-s to about mPa-s. Preferably a hydrogel made from PVA has a viscosity of about 20 mPa-s to about 40 mPa-s. Most preferably a hydrogel made from PVA has a viscosity of about 28 mPa-s.

Viscosity may be measured using a 4% aqueous solution at 200 degrees C. determined by Brookfield synchronized-motor rotary type.

The polymer of the hydrogel may be about 7.5% to PAAm to about 20% PAAm (w/w). A hydrogel made from about 7.5% PAAm to about 20% PAAm (w/w) may have a coefficient of friction of about 0.1 or less. Hydrogels made from polymers comprising about 7.5% PAAm have a low friction coefficient (e.g., about 0.1 or less). The polymer of the hydrogel may be about 15% PAAm (w/w). Hydrogels made from a polymer comprising about 15% PAAm have a low friction coefficient (e.g., about 0.1 or less) and are strong when combined with a mesh. Thus, for example, the polymer of the hydrogel may be about 92.5% water to about 80% water (w/w) and about 7.5% PAAm to about 20% PAAm (w/w).

The polymer of the hydrogel may be a HEMA copolymer. The polymer may be about 40% to about 98% HEMA copolymer (w/w). A hydrogel made from about 40% HEMA to about 98% HEMA (w/w) may have a coefficient of friction of about 0.1 or less. In embodiments where the HEMA-copolymer is HEMA-MAA, the polymer may comprise about 90% to about 98% HEMA-MAA (w/w). Thus, for example, the polymer may be about 90% to about 98% HEMA-MAA (w/w) and about 10% to about 2% water (w/w). In embodiments where the HEMA-copolymer is HEMA-VP, the polymer may comprise about 90% to about 40% HEMA-VP (w/w). Thus, for example, the polymer may be about 90% to about 40% HEMA-VP (w/w) and about 10% to about 60% water (w/w).

Once the polymer of the hydrogel has been created (e.g., by mixing the polymer (powder) with water, and then drying the mixture), a liquid may be added to the polymer (e.g., the dried mixture) to create a hydrogel. The liquid of the hydrogel or hydrogel membrane may be water (e.g., deionised water), a water-based lubricant or an oil-based lubricant. A water-based lubricant may be an aqueous solution. A water-based lubricant may not comprise an oil or a silicone-containing lubricant. The liquid (e.g., water) of the hydrogel acts as a lubricant and thus makes the hydrogel lubricious. The highly absorbent nature of the hydrogel may cause the device to absorb natural lubricants from within a birth canal.

The presence of the liquid (e.g., water) in the hydrogel causes the hydrophilic polymer of the hydrogel to swell. The swell ratio of the polymer may be between 2-20:1.

In one embodiment, the hydrogel comprises a 3D network of a hydrophilic polymer (e.g., PVA, or a HEMA copolymer) and water (e.g., deionised water) to encourage swelling of the 3D network. Thus, the hydrogel may comprise a 3D network of PVA and water (e.g., deionised water).

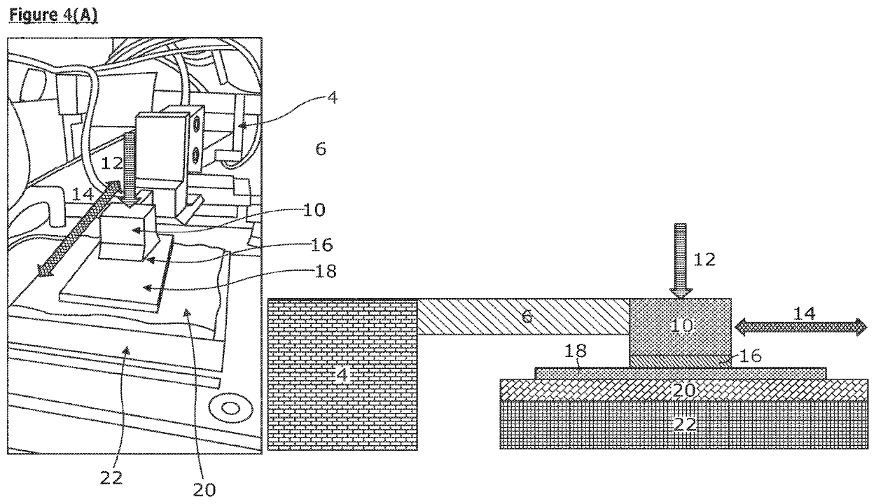

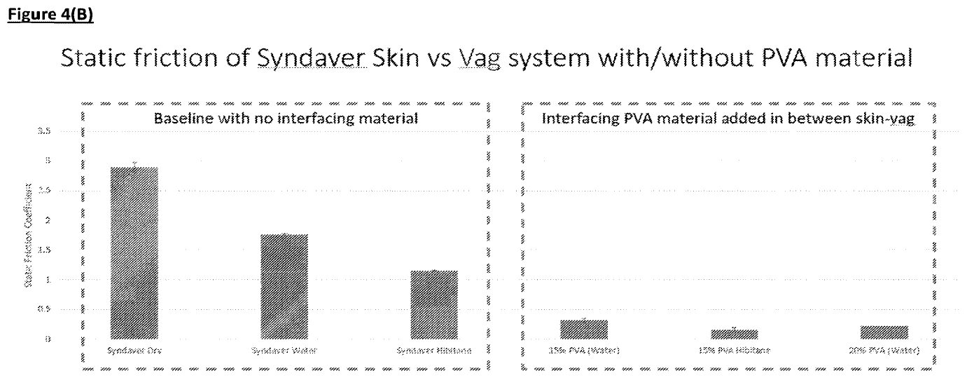

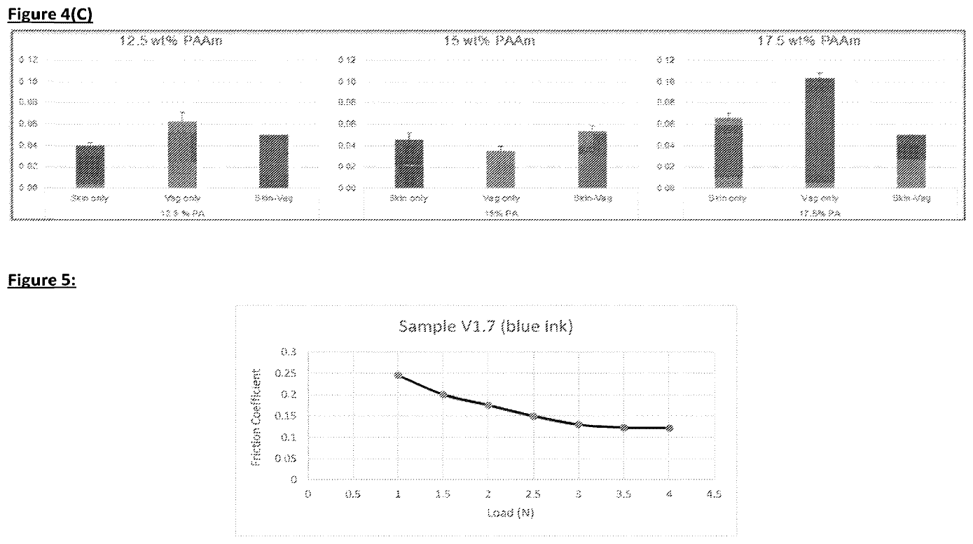

The friction coefficient of the device may be calculated using a ‘2D’-bench test setup (see FIG. 4A).

The ‘2D’ bench set up (biotribometer [BTM, PS Instruments, London]) measures the frictional force generated during sliding contact between a synthetic skin, which is used to simulate the skin of a baby's head (141500, Syndaver, Tampa FL, USA), and synthetic vaginal tissue, which is used to simulate tissue of the birth canal (141690, Syndaver, Tampa FL, USA), optionally with an embodiment of the device as an interface between the synthetic tissue and synthetic skin. The experimental conditions used in the ‘2D’ setup are as follows:

| | |
|---|---|
| Setup | BTM, PCS Instruments, UK |
| Skin tissue | Syndaver skin tissue (141500) |
| | [2 mm in thickness] |
| Birth canal tissue | Syndaver vaginal tissue (141690) |
| | [4 mm in thickness] |
| Load | IN |
| Velocity | 1 mm s⁻¹ |
| Motion amplitude | 10 mm |
| Output metric | Static coefficient of friction |

The output of the ‘2D’ setup is the static coefficient of friction.

The ‘3D’-birth-simulator setup measures resistance force and movement distance as an artificial head covered with SynDaver® foetal skin (141500, Syndaver, Tampa FL, USA [5 mm in thickness]), which may in turn be covered with an embodiment of the device, is pushed through an artificial birth canal lined with SynDaver® vaginal tissue (141690, Syndaver, Tampa FL, USA [20 mm in thickness]). In other words, the 3D set up simulates and measures the amount of energy required from a mother to push a baby out of a birth canal with/without an embodiment of the device. The artificial head may be pushed through the birth canal for 70 mm, at a speed of 1 mm/s. The output measured is the instantaneous force in N and the total work done in J. These output values may then be used to calculate the total loss of energy using the following equation:

$${E}_{loss} = {\sum }_{i = 1}^{n} ( {x}_{i} - {x}_{i - 1} ) {F}_{i}$$

Where xᵢ represent the cumulative displacement of the “fetal head” into the “birth canal” at step i. The total amount of step n is 7000, where at each step or every 0.01 s the actual cumulative displacement and the instantaneous force Fᵢ is measured by the system.

An embodiment of the device, particularly the hydrogel or the hydrogel membrane, has a low coefficient of friction. A low coefficient of friction can refer to a value of about 0.5 or less, about 0.4 or less, about 0.3 or less, about 0.2 or less, or about 0.1 or less (according to a 2D bench setup).

The coefficient of friction of the hydrogel or the hydrogel membrane of an embodiment of the device may be about 1 or less, about 0.9 or less, about 0.8 or less, about 0.7 or less, about 0.6 or less, about 0.5 or less, about 0.4 or less, about 0.3 or less, about 0.2 or about less, or about 0.1 or less. A device comprising a PAAm hydrogel or hydrogel membrane may have a friction coefficient of about 1 or less, about 0.9 or less, about 0.8 or less, about 0.7 or less, about 0.6 or less, about 0.5 or less, about 0.4 or less, about 0.3 or less, about 0.2 or about less, or about 0.1 or less. A device comprising a PVA hydrogel or hydrogel membrane may have a coefficient of friction of about 1 or less, about 0.9 or less, about 0.8 or less, about 0.7 or less, about 0.6 or less, about 0.5 or less, about 0.4 or less, about 0.3 or less, about 0.2 or about less, or about 0.1 or less. A device comprising a PEG hydrogel or hydrogel membrane may have a friction coefficient of about 1 or less, about 0.9 or less, about 0.8 or less, about 0.7 or less, about 0.6 or less, about 0.5 or less, about 0.4 or less, about 0.3 or less, about 0.2 or about less, or about 0.1 or less. A device comprising a HEMA copolymer hydrogel or hydrogel membrane may have a coefficient of friction of about 1 or less, about 0.9 or less, about 0.8 or less, about 0.7 or less, about 0.6 or less, about 0.5 or less, about 0.4 or less, about 0.3 or less, about 0.2 or about less, or about 0.1 or less. A device comprising a PDMS hydrogel or hydrogel membrane may have a coefficient of friction of about 1 or less, about 0.9 or less, about 0.8 or less, about 0.7 or less, about 0.6 or less, about 0.5 or less, about 0.4 or less, about 0.3 or less, about 0.2 or about less, or about 0.1 or less. The coefficient of friction may be calculated using a ‘2D’ bench test setup.

The skilled person will appreciate that the thicker the hydrogel layer is, the more difficult it will be to place the cap on the head of a baby in a birth canal as well as the more difficult it will be to deliver the baby. However, the thinner the hydrogel layer is, the more prone that it is to being torn. Consequently, the hydrogel may comprise synthetic polymer fibres (e.g., polyamide fibres, such as nylon). The synthetic polymer fibres may be embedded or immersed within the hydrogel. The synthetic polymer fibres may reinforce the hydrogel without substantially increasing the overall thickness of the hydrogel. The term substantially can refer to an increase of less than about 30%, less than about 25%, less than about 20%, less than about 15%, less than about 10%, less than about 5%, or less than about 1%. The term substantially can refer to no increase in the overall thickness of the hydrogel.

The synthetic polymer fibres may or may not be woven. The synthetic polymer fibres (e.g., polyamide fibres, such as nylon) may form a mesh. The synthetic polymer fibres may form at least one layer, or two or more layers of synthetic polymer fibres. The layer(s) of synthetic polymer fibres may or may not be porous. The layer of synthetic polymer may be a mesh, e.g., a knitted mesh. The mesh may be an open mesh, a filter mesh, a woven mesh, or a warp knit mesh. The mesh may be warp knitted as this provides a smooth surface while maintaining reasonable flexibility at a thin thickness. The synthetic polymer fibres do not dissolve in the hydrogel or a liquid, such as water.

Advantageously, the synthetic polymer fibres enable the hydrogel of the device to have minimal thickness while maintaining low friction and good flexibility/tensile strength. The synthetic polymer fibres also prevent the network of hydrophilic polymer from deforming due to excess swelling. Thus, the synthetic polymer fibres enable the hydrogel to retain a well-defined structure.

The pores of the mesh or layer of synthetic polymer fibre may have a diameter (or largest length) of about 0.5 mm to about 20 mm. Pores within this range are capable of reinforcing the hydrogel and preventing it from excessive swelling. The pores may have a diameter (or largest length) of about 1.0 mm. The fibres of the mesh may be between about 0.05 mm and 0.4 mm in thickness/diameter. The fibres of the mesh may be about 0.2 mm in diameter/thickness. Preferably, the mesh (e.g., polyamide) has a 0.4 mm by 0.4 mm pore size and fibres of about 0.2 mm thickness.

The synthetic polymer of the synthetic polymer fibres may be hydrophilic or hygroscopic. Thus, the synthetic polymer fibres may form hydrogen bonds with water molecules in the hydrogel. The water molecules may also form hydrogen bonds with the hydrophilic polymer of the hydrogel. Thus, hydrogen bonds help to prevent delamination or separation of the hydrogel from the synthetic polymer fibres when the device is placed under stress. The synthetic polymer of the synthetic polymer fibres may be a selection of one or more from the group consisting of: polyamide (e.g., nylon), a polyacrylonitrile, a polyester, a polypropylene, a polybutester, a polyurea, and a polyurethane. Preferably the synthetic polymer is a polyamide. More preferably the synthetic polymer is nylon or elastane (i.e., a copolymer of polyether and polyurea). More preferably the synthetic polymer fibres form at least one layer of a polyamide (e.g., nylon). The at least one layer of the porous polyamide may be porous.

In one embodiment, the synthetic polymer fibres are made from or comprise a polyamide (e.g., nylon), and the hydrogel is made from PVA and water (e.g., deionised water). In another embodiment, the synthetic polymer fibres are made from or comprise a polyamide (e.g., nylon), and the hydrogel is made from or comprises a HEMA copolymer and water (e.g., deionised water). In another embodiment, the synthetic polymer fibres are made from or comprise a polyamide (e.g., nylon), and the hydrogel is made from or comprises a PAAm copolymer and water (e.g., deionised water). The synthetic polymer fibres may be made into a mesh by warp knitting.

The hydrogel crown or the hydrogel membrane may be less than 2 mm, less than 1.5 mm, less than 1.0 mm, or less than 0.5 mm in thickness. The hydrogel crown may be greater than 0.01 mm, greater than 0.05 mm, greater than 0.1 mm, greater than 0.2 mm, greater than 0.25 mm, or greater than 0.3 mm in thickness. Preferably, the hydrogel crown or hydrogel membrane is between about 0.05 mm and 10 mm in thickness, between about 0.1 mm and 2 mm in thickness, or between about 0.05 mm and 0.5 mm in thickness. The hydrogel crown or the hydrogel membrane may be of uniform thickness.

The bulbous rim is greater in thickness than the crown of the device or a wall (the remainder) of the hydrogel membrane. The bulbous rim may be at least about 5-fold, at least about 10-fold, at least about 20-fold thicker, at least about 50-fold thicker, or at least about 100-fold thicker than the crown, or the wall of the hydrogel membrane, preferably the bulbous rim is about 5-fold to about 10-fold thicker, most preferably the rim is about 5-fold thicker.

The crown may be between 0.5 mm and 2 mm in thickness and made from PVA hydrogel. The crown may be between 0.5 mm and 2 mm in thickness and made from 5% to 20% PVA hydrogel. The crown may be between 0.5 mm and 2 mm in thickness and made from about 15% PVA hydrogel. The hydrogel membrane may be between 0.5 mm and 2 mm in thickness and made from PVA hydrogel. The membrane may be between 0.5 mm and 2 mm in thickness and made from 5% to 20% PVA hydrogel. The hydrogel membrane may be between 0.5 mm and 2 mm in thickness and made from about 15% PVA hydrogel.

One surface of the device may comprise a high friction material. The high friction material may be on one surface of the crown or the membrane and/or the bulbous rim. The high friction material may be a material referred to herein. For example, the high friction may be one or more materials selected from the group consisting of: rubber, silicone, polyethylene, polyurethane, nylon, polydimethylsiloxane, polyvinylchloride, polyethersulfone, polytetrafluoroethylene, polyetherimide, polycarbonate, polysulfone, polyetheretherketone and polypropylene. In embodiments in which the device is reinforced with synthetic polymer fibres, the high friction material may protrude from within the hydrogel to an outer surface, e.g., an outer surface of the crown, the sleeve, the cap, the membrane, and/or the bulbous rim. The high friction material may be present on an inner surface of the device (i.e., a surface, which in use, is contacted by the baby). Thus, while in a birth canal, the outer surface of the device is more likely to slide against the birth canal than the inner surface of the device sliding against the baby. Consequently, the device may aid delivery of a baby, preferably while simultaneously exiting the birth canal. In another embodiment, the high friction material may be present on an outer surface of the device (i.e., a surface, which in use, is contacted by the birth canal). Thus, while in a birth canal, the baby is more likely to slide against the inner surface of the device than the outer surface of the device sliding against the birth canal. Consequently, the device may aid delivery of a baby, preferably without simultaneously exiting the birth canal.

According to a second aspect of the disclosure, there is provided a method of making a device for assisting childbirth, the method comprising:
moulding a hydrogel solution comprising synthetic polymer fibres into a device for assisting childbirth.

According to another aspect of the disclosure, there is provided a method of making a device for assisting childbirth, the method comprising:
moulding a hydrogel solution into a device for assisting childbirth.

In one embodiment, the hydrogel solution comprises synthetic polymer fibres. The method may be for making a device according to the disclosure.

Moulding may comprise casting the hydrogel solution to create a cast comprising the hydrogel solution and synthetic polymer fibres; initiating hydrogel formation; and then curing the cast to form a device for assisting childbirth. Moulding may be injection moulding, cast moulding, or compression moulding.

The hydrogel solution may be created by mixing a powder of hydrophilic polymer in a liquid to create a mixture. The liquid may be water, preferably deionised water.

The “initiating” step may comprise performing a heat-cooling method. In a heat-cooling method, the cast/mixture may be heated until a clear, transparent, viscous solution is created. The cast/mixture may be mixed during the heating step.

The curing step may comprise waiting for the cast to cure (or cool) at room temperature. The curing step may be performed at about 10° C. to about 25° C. Preferably the curing step is performed at about 20° C. The curing step may comprise using UV light.

The curing step may be at least about 10 minutes, at least about 20 minutes or at least about 30 minutes long. Preferably the curing step is at least about 30 minutes long. The cooling step may be at least about 10 minutes to about 50 minutes, or about 20 minutes to about 40 minutes.

Thus, the curing step may be performed at about 10° C. to about 25° C. for about 20 minutes to about 40 minutes.

The method may further comprise performing one, two, three or four freeze-thaw cycle(s), preferably after the curing step. The method may further comprise performing two or three freeze-thaw cycles after the curing step. The freeze-thaw cycles influence the mechanical properties of the hydrogel.

A freeze-thaw cycle may comprise freezing the device at about −35° C. to about −15° C. for at least about 12 hours, followed by thawing the device at about 4° C. for at least about 4 hours. A freeze-thaw cycle may comprise freezing the device at about −25° C. for at least about 12 hours, followed by thawing the device at about 4° C. for about 4 hours

The method may further comprise hydrating the device, optionally after a freeze-thawing step. Hydrating the device may be performed by placing it in a liquid, such as water (e.g., deionised water) or an aqueous solution. The hydrating step may be performed (e.g., with water) for about 12 to 48 hours, for about 18 to 48 hours, or about 24 hours. Preferably the hydrating step is performed in water or aqueous solution for a maximum of about 48 hours. Preferably the hydrating step is performed at about 10° C. to about 25° C. for about 24 hours.

The method according to the second aspect may be used to create a device (e.g., a cap, a sleeve or a hydrogel membrane) according to the disclosure.

According to a third aspect, there is provided a device made or capable of being made by a method according to the disclosure.

According to a fourth aspect, there is provided a method of assisting childbirth by a pregnant subject, particularly by preventing obstructed labour, the method comprising:
placing a device according to an aspect of the disclosure on the head of a baby within the birth canal of the subject so as to assist childbirth; or using a device according to the disclosure to line at least part of a birth canal/or cover at least part of a baby in a birth canal during childbirth.

Placing the device on the head of a baby while in the birth canal or lining at least part of a birth canal or covering at least part of a baby in a birth canal breaks contact between the baby or the baby's head and the mucosal tissue of the birth canal. Mechanical pressure placed on the hydrogel during labour causes the hydrogel to release its liquid (e.g., water) and thus generate a lubricating fluid film. The film is produced without requiring motion between baby and mother. This type of lubrication, which depends on the low compliance of the hydrogel and its ability to contain an interfacial water film even in the absence of relative motion, results in a stable and low friction interaction between the device and the tissue of the birth canal. Thus, a method of the disclosure enables one to assist childbirth or prevent obstructed labour by reducing the level of friction between the baby and the birth canal.

Placing the device on the head of a baby within the birth canal may comprise folding the crown or the sleeve such that there is a double layer of reinforced hydrogel between the baby's head and the birth canal. Lining at least part of a birth canal or covering at least part of a baby within the birth canal may comprise folding the hydrogel membrane such that there is a double layer of reinforced hydrogel between the baby's head and the birth canal.

The term “childbirth” herein refers to birth through the birth canal or unassisted vaginal delivery. Childbirth does not comprise childbirth through caesarean section. Thus, childbirth for example includes giving birth to a child in the breech position or exhibiting cephalic presentation (head-first). A sleeve according to the disclosure may be used to deliver a baby in the breech position.

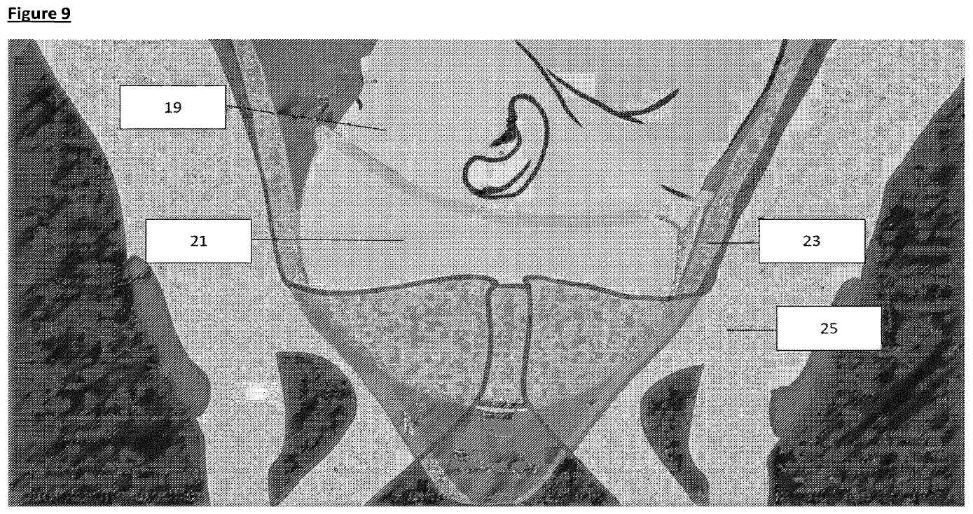

For childbirth to occur through the birth canal (23), the baby, particularly the head (19) must pass through the pelvis (25) of the pregnant subject (see FIG. 9). FIG. 9 shows the head of a baby (19) covered in a cap (21) according to the disclosure situated in the birth canal (23).

The pelvic bone can be divided into three regions, (1) the inlet (superior aperture), (2) the mid-cavity, and (3) the outlet (inferior aperture). The inlet is wider in the transverse direction than anteroposterior direction, the mid-cavity is circular, whereas the outlet is wider in the anteroposterior direction.

In one embodiment, the method comprises placing the device on the head of the baby while the pregnant subject is in the second stage of labour. Thus, the method according to the fourth/third aspect comprises inserting the device into the birth canal of the subject and placing it on the head (and optionally the body) of the baby. Alternatively, the method according to the fourth aspect may comprise inserting an embodiment of the device into the birth canal of the subject so as to prevent direct contact between the skin of the baby in the birth canal and the mucosal lining of the birth canal. However, the method may include distributing a liquid lubricant over the outer surface of the cap prior to insertion into the birth canal or while it is in the birth canal.

An embodiment of the method may comprise placing the device (e.g., the sleeve or the cap) on the head of a baby exhibiting cephalic presentation (head-first).

The foetal head is the widest part to get through the passage during the labour process, once the head is out of the passage, the rest of the labour is relatively easy to deliver. An embodiment of the device may therefore be placed on the baby's head (and optionally the baby's body) to assist passage of the baby through the pelvis. Thus, the device may be placed on the baby once the widest part of the baby's head is in the inlet, the mid cavity, or the outlet. The device may be placed on the baby once the widest part of the baby's head has passed through the inlet or the mid cavity.

Station levels are used to assess the descent of the foetus through the birth canal, where station 0 is represented by the horizontal plane formed by the ischial spine in the mother's pelvic mid-cavity. The device may be placed on the head of the baby once descent to station level 0 or more has occurred. Thus, the device may be placed on the head of the baby once it has descended to station level 0 or more, 1 or more, 2 or more or 3 or more. Most preferably, the device is placed on the head of the baby once it has descended to station level 1 or more.

An embodiment of the method may comprise placing the device on the head of a baby during cephalic presentation.

The head of the baby comprises plates connected by sutures. The sutures between the bone plates are soft, during labour. Thus, the skull shape may change under pressure (moulding) to fit through the passage. Bone plates, may for example, overlap with each other during moulding.

In addition to the shape of the baby's head potentially changing during labour, the foetal attitude may vary. The attitude refers to the posture of the baby (i.e., flexed, deflexed, or extended). The attitude of the baby will thus determine how large the widest diameter will be, which in turn, affects the ease with which the baby is delivered.

The baby's head is not always completely flexed when it enters the pelvis. As the head descends into the narrower mid-pelvis, flexion occurs. The foetal chin tucks in towards its chest so the presenting diameter is smaller to allow easier passage through the mother's pelvic bone.

The widest diameter of the baby's head may be suboccipitobregmatic (well flexed), occipitofrontal (partially extended or deflexed), occipitomental (extended brow presentation) or submentobregmatic (hyperextended face presentation).

The device may be placed on the head of a baby while it is well flexed. The device may be placed on the head of a baby while it is partially extended or deflexed. The device may be placed on the head of a baby while it is in extended brow presentation. The device may be placed on the head of a baby while it is in hyperextended face presentation.

In one embodiment, the device is placed on the head of a baby while it is suboccipitobregmatic (well flexed) or submentobregmatic (hyperextended face presentation).

The head of the baby may enter the pelvic inlet and exit the outlet in a different position. The baby may present in the outlet in an occipito-anterior (OA) position, such as a right occipito-anterior position, a straight occipito-anterior position, or a left occipito-anterior position. The baby may present in the outlet in an occipito-transverse (OT) position, such as a right occipito-transverse position, a straight occipito-transverse position, or a left occipito-transverse position. The baby may present in the outlet in an occipito-posterior (OP) position, such as a right occipito-posterior position, a straight occipito-posterior position, or a left occipito-posterior position. Preferably the baby presents in an OA position.

Thus, the device may be placed on a baby's head in an OA position, an OT position or an OP position.

An embodiment of the method may comprise removing the device from the baby as soon as childbirth has occurred (i.e., once the baby has been delivered).

According to a fifth aspect, there is provided a use of an embodiment of the device to assist childbirth and/or prevent obstructed labour during childbirth.

In another aspect, there is provided an embodiment of the device for use in preventing obstructed labour during childbirth.

According to a sixth aspect, there is provided a use of a hydrogel to assist childbirth and/or prevent obstructed labour during childbirth.

In an embodiment, the hydrogel is a hydrogel membrane. The hydrogel membrane may be a hydrogel membrane referred to herein.

An embodiment may comprise using one or more separate hydrogel membranes. Thus, an embodiment may comprise using one or more, two or more, three or more, four or more, or five or more separate hydrogel membranes. An embodiment may comprise using an embodiment of the device to assist childbirth and/or prevent obstructed labour during childbirth.

In another embodiment, there is provided a hydrogel for use in preventing obstructed labour during childbirth.

A “device” referred to herein may be a cap or a sleeve according to the disclosure.

The term “hydrogel” can refer to a hydrogel membrane, such as the membrane referred to herein.

A “hydrogel membrane” can refer to a layer of insoluble material made of or comprising a hydrogel.

The term “lining” can refer to covering. Thus, the hydrogel membrane according to the disclosure may be for covering at least part of a birth canal during childbirth, for example, covering a total surface area of at least about 1 cm².

The term “comprising” may refer to “consisting of” or “consisting essentially of”.

All of the embodiments and features described herein (including any accompanying claims, abstract and drawings), and/or all of the steps of any method or process so disclosed, may be combined with any of the above aspects or embodiments in any combination, unless stated otherwise with reference to a specific combinations, for example, combinations where at least some of such features and/or steps are mutually exclusive.

For a better understanding of the disclosure, and to show how embodiments of the disclosure may be put into effect, reference will now be made, by way of example, to the accompanying drawings, in which:

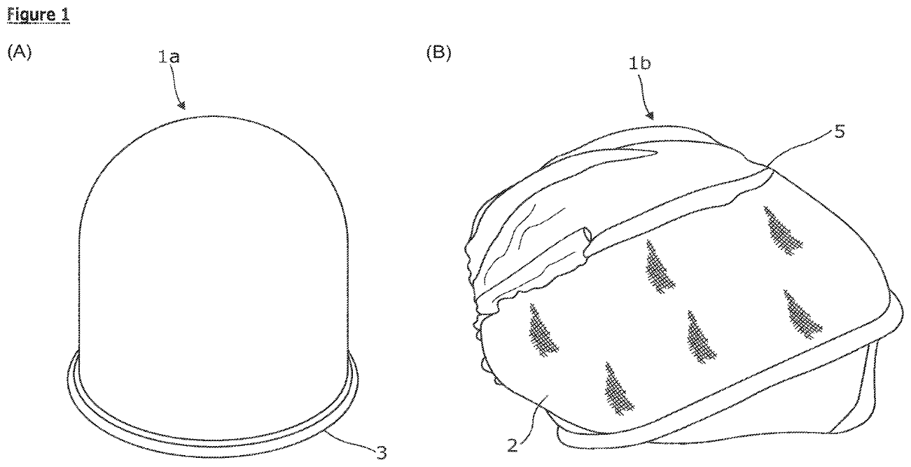

FIG. 1 is (A) a CAD of a cap according to an embodiment; and (B) a picture of a cap on the head of a training dummy.

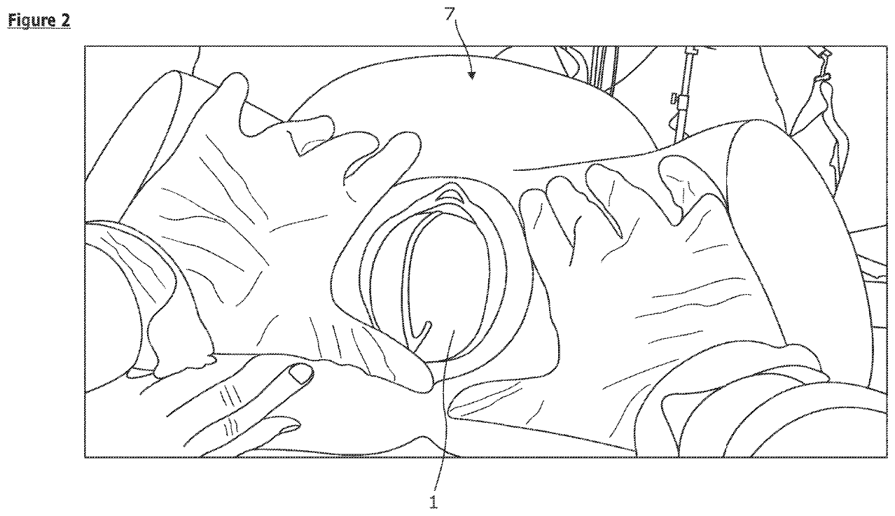

FIG. 2 is a picture of the head of a training dummy emerging from the birth canal of a birthing dummy while wearing a cap according to an embodiment.

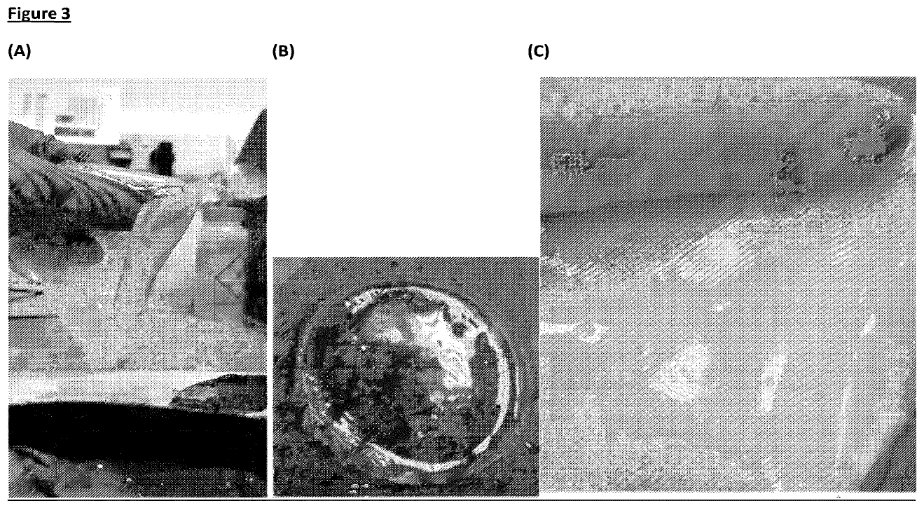

FIG. 3 is (A) a picture of a PAAm hydrogel sheet reinforced with a nylon warp knit mesh, (B) a picture of a HEMA copolymer (HEMA-MAA) hydrogel dish without a mesh, and (C) a PVA hydrogel sheet reinforced with a nylon warp knit mesh.

FIG. 4 (A) is a schematic overview of a test setup used to measure the friction coefficient of SynDaver® synthetic vaginal tissue vs synthetic skin tissue with the addition of a friction reducing material; (B) shows the experimental results obtained on the 2D bench setup under different conditions, “Syndaver dry” refers to SynDaver® skin vs vaginal tissue under padded dry conditions, “Syndaver wet” refers to SynDaver® skin vs vaginal tissue with excess water, “SynDaver® hibitane” refers to SynDaver® skin vs vaginal tissue with the lubricant, Hibitane™. “15% PVA (water)” refers to a hydrogel interface made with 15% PVA and water, “20% PVA (water)” refers to a hydrogel interface made with 20% PVA and water, “15% PVA hibitane” refers to a hydrogel interface made with 15% PVA and water and coated in the lubricant Hibitane™; and (C) shows the experimental results obtained from the 2D bench setup using various PAAm hydrogels—“Skin only” refers to the sliding surface was SynDaver® skin vs PAAm hydrogel, “Vag only” refers to the sliding surface was SynDaver® vaginal tissue vs PAAm hydrogel, “Skin-Vag” refers to the sliding surface was SynDaver® Skin vs vaginal tissue with PAAm hydrogel sandwiched between.

FIG. 5 shows the effect of loading force applied on the static friction coefficient of a wet hydrogel sample (15% PVA) tested using a 2D setup (SynDaver® vaginal/skin tissue).

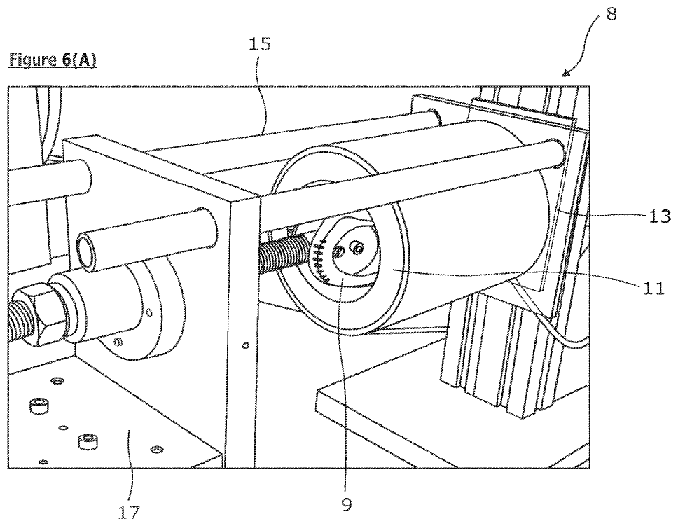

FIG. 6 shows (A) a ‘3D’-birth-simulator setup with an artificial head covered with SynDaver® foetal skin, positioned in an artificial birth canal lined with SynDaver® vaginal tissue, (B) a close-up of the ‘3D’-birth-simulator setup with prototype visible between head and canal.

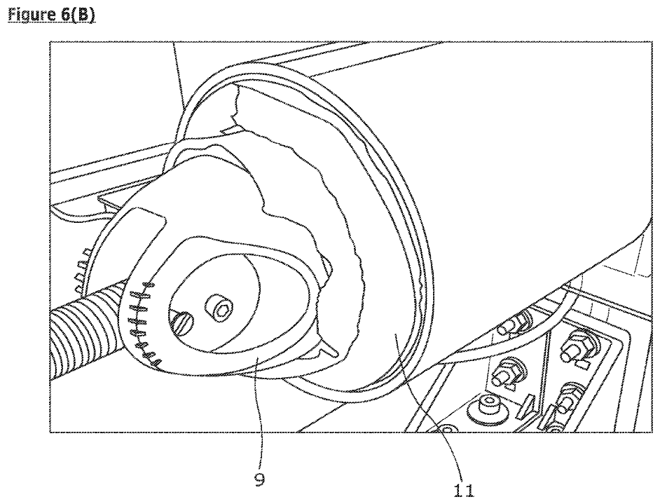

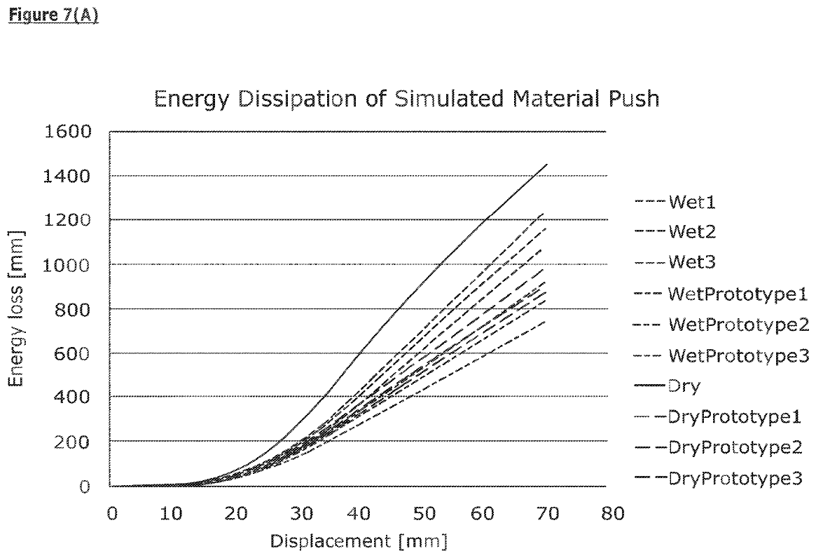

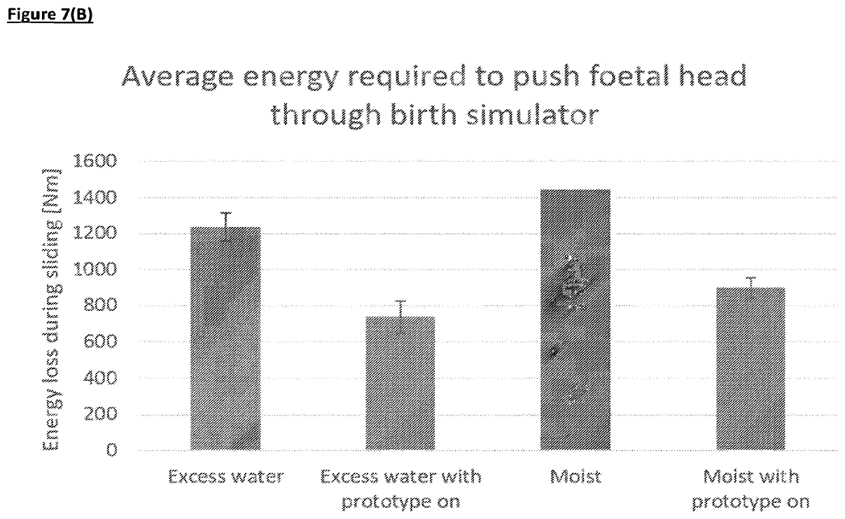

FIG. 7 shows (A) the cumulative energy dissipation results for a simulated maternal push, where the foetal head was pushed through the birth canal of a 3D-birth simulator at a fixed speed and over the same distance, under various experiment conditions repeated three times each, “Wet” refers to a birth canal with excess water and no prototype, “Dry” refers to a moist birth canal padded dry with paper towel with no prototype, “Wet Prototype” refers to a birth canal with excess water and prototype on the foetal head, “Dry Prototype” refers to a moist birth canal with the prototype on the foetal head and (B) a summary of the results of FIG. 7(A).

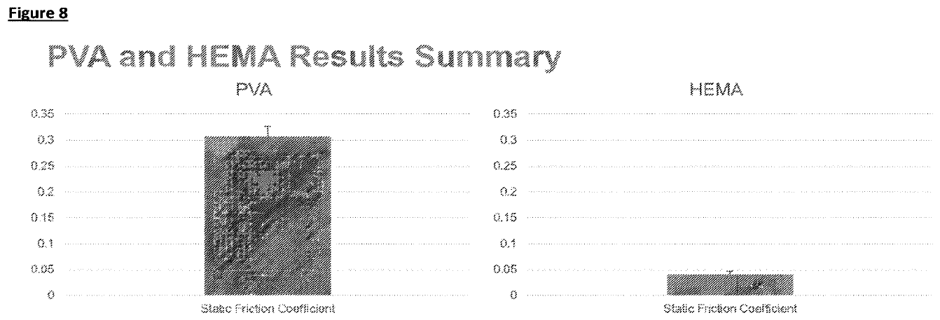

FIG. 8 shows the static friction coefficient for a PVA hydrogel and a HEMA-MAA hydrogel obtained on the 2D bench setup of FIG. 4.

FIG. 9 is an illustration of a device according to an embodiment placed on a baby's head while inside a birth canal during second stage labour.

FIG. 10 shows (A) a perspective view of a hydrogel comprising a reinforcing mesh, (B) a plan view of a hydrogel comprising a reinforcing mesh, and (C) a picture of a warp mesh.

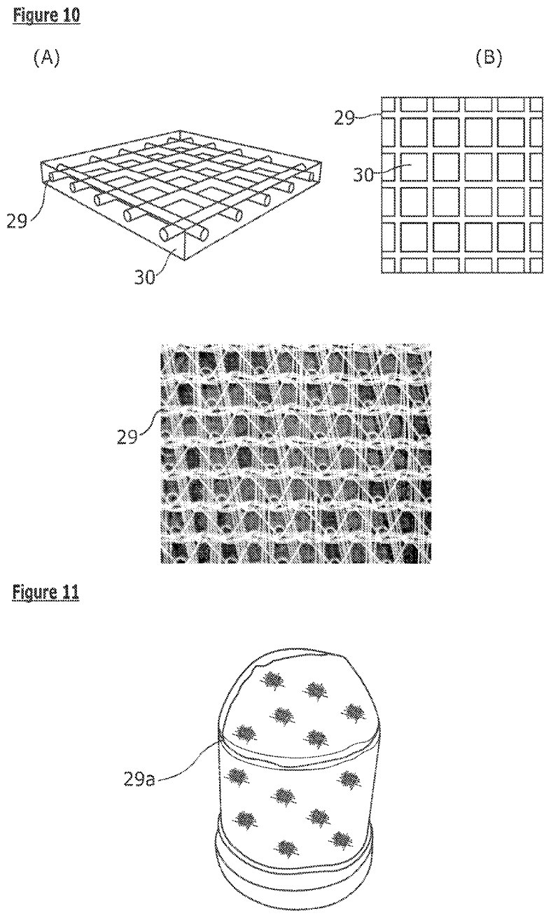

FIG. 11 is a picture of a nylon mesh (29a) on a mould (before the mesh moulded in a liquid of hydrophilic polymer).

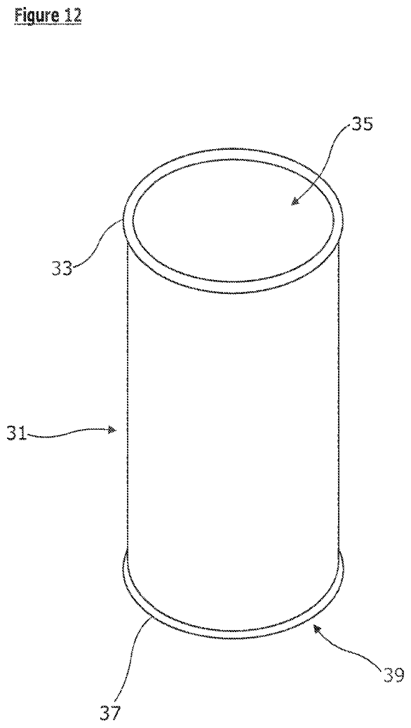

FIG. 12 is a picture of a sleeve according to one embodiment of the disclosure.

### EXAMPLES

FIG. 1 is (A) a CAD of a cap (1a) according to one embodiment of the disclosure. The cap (1a) comprises an opening (not shown) defined by a bulbous rim (3). In FIG. 1(B), the cap (1b) is made from a PVA hydrogel reinforced with a nylon mesh (2). The cap has been made by attaching (e.g., sewing) several sheets of reinforced hydrogel together. Thus, the cap also comprises a seam (5).

Referring to FIG. 2, there is shown a birthing dummy (7) being used by a childbirth trainer. The head of a training dummy within the birth canal of the birthing dummy is covered by a cap (1) according to an embodiment. The cap is inserted into the birth canal and on to the head of the baby before it emerges so as to assist delivery of the training dummy.

Referring to FIG. 3(A), there is shown a picture of a PAAm hydrogel sheet that contains a nylon mesh. FIG. 3(B) is a picture of a hydrogel made from a HEMA based copolymer (HEMA-MAA). FIG. 3(C) is a picture of a PVA hydrogel that contains a nylon mesh. The glossy appearance of the hydrogel is caused by water stored within the pores of the hydrogel and the mesh. In use, the hydrogel sheet is used to create a cap according to an embodiment. Alternatives and modifications within the scope of the disclosure will be apparent to the skilled person. For example, the hydrogel may be made from a polyacrylamide, polyvinyl alcohol (PVA), polyacrylamide (PAAm), HEMA-based copolymer, polyethylene glycol (PEG), polydimethylsiloxane (PDMS) or mixtures thereof; contain a water-based lubricant; and the synthetic polymer fibres may form a non-porous layer.

Referring to FIG. 4(A), there is shown a ‘2D’ bench setup for measuring sliding friction. Tribological testing to determine the friction coefficient of a hydrogel is carried out using a Biotribometer machine (PCS Instruments). The machine features a moving top component that slides against a fixed bottom plate with a specified load applied. The test involves fixing two materials to the top arm (6) and the bottom plate (22) in order to determine the friction coefficient of the relevant tribosystem comprising the two materials. For example, a skin mimic (SynDaver®) (16) and a vagina mimicking material (20) from SynDaver®. A vagina mimicking material (20) from SynDaver® is applied to the bottom plate (22) while a skin mimic (16) is applied to the specimen holder (10) to simulate the in vivo scenario more closely. Hydrogel samples (18) are then placed between the two skin mimics (16, 20) and allowed to slide freely. An applied load (12) of, for example, 1N over a stroke length of 20 mm at 0.5 mm/s speed is used to evaluate the effective friction coefficient of the system. (4) corresponds to the actuation system and (6) corresponds to the actuation arm.

FIG. 4(B) is a summary of experimental results obtained using the 2D′ bench setup. The results clearly show that placing a hydrogel, such as PVA, between the “vaginal tissue” and “skin”, significantly reduces static/sliding friction.

FIG. 5 shows that the greater the force applied to the specimen tissue the smaller the frictional coefficient becomes in the presence of a hydrogel according to an embodiment.

FIG. 6(A) and 6(B) shows a 3D′-birth-simulator setup (8) with an artificial head (9) covered with synthetic skin tissue positioned in a birth canal (11) lined with synthetic vaginal tissue. The simulator is used to measure the amount of energy required to slide a baby's head (9) through a birth canal (11), as well as enable a user to measure average steady state force. (13) corresponds to the location of a force transducer (not shown), (15) corresponds to two removable alignment beams, and (17) corresponds to an actuation platform

FIGS. 7(A) and 7(B) show that introduction of various prototypes into the 3D′-birth-simulator setup reduces the energy required to slide the head through the birth canal by about 40%.

FIG. 8 shows that static friction coefficient of a hydrogel made with the PVA and a separate hydrogel made with HEMA-MAA (poly(2-hydroxyethyl methacrylate/methacrylic acid). The static friction coefficients were obtained using the 2D′ bench setup described above. The HEMA-based hydrogel has a coefficient of friction which is more than 6-fold smaller than that of PVA.

FIGS. 10(A) and 10(B) show a nylon mesh (29) embedded in a hydrogel (30). FIG. 10(C) shows a picture of a nylon warp knit mesh.

FIG. 12 shows a sleeve (31) according to one embodiment of the disclosure. The sleeve (31) comprises a first opening (35) and a second opening (39). The first opening (35) is defined by a first bulbous rim (33) and the second opening (39) is defined by a second bulbous rim (37).

### Example 1—Manufacturing a 15% PVA Hydrogel Cap Comprising a Mesh

### The Materials

Poly(vinyl alcohol) “PVA” (146,000-186,000 g mol⁻¹, CAS: 9002-89-5) and deionised water were supplied by Sigma-Aldrich UK. A nylon (warp knitted) mesh. A beaker, made from borosilicate glass, was used as it can withstand the high temperature and pressure that it will be exposed to.

An example of the constituent amounts is listed in Table 1. The total weight can be adjusted depending on the sizes of the moulded product.

TABLE 1
Calculated weights for PVA, to be scaled as desired.

| PVA 15% | Weight |
|---|---|
| PVA powder | 15 (g) |
| DI water | 85 (g) |

### Protocol

1. Place the nylon (warp knitted) mesh in a mould.
2. Measure DI water and PVA powder constituents and place them into separate beakers.
3. Place the 15% PVA mixture into a high temperature pressure cooker or equivalent and set to ‘High’ for 1 hour. Stop the cooker every 20 mins and mix manually.
4. This means after 20 mins, stop the cooker, take out the beaker, mix the solution manually and thoroughly i.e., with a metal spatula or something equivalent (not plastic as it might melt) and put it back into the cooker, set it on ‘High’ for another 20 mins, take it out, mix it manually, and so on, for around 1 hour total cooking time. After 1 hour cooking time, the final solution should be clear, transparent (all PVA particles should have dissolved) and highly viscous. If it is not, continue repeating the cooking and mixing cycle until the correct solution is achieved. If the solution is a jelly that is either because the solution has not been heated up enough to where all the PVA particles have dissolved, or the manual mixing was not sufficient.
5. Take care to avoid excessive evaporation during the process; if using a conical flask, the screw cap should be loosely fitted and if using an open flask, aluminium foil should be fitted to cover the flask opening.
6. While boiling and straight out of the pressure cooker, pour 15% PVA into the mould and close the mould.
7. Wait 30 mins for it to cool and settle.
8. Place in a freezer (approx. −25° C.) for 18 h (overnight).
9. Thaw at 4° C. (in a fridge) for 4 hours. Bring sample to room temperature (1 hour).
10. Demould and hydrate the cap in deionised (DI) water for about 24 hours.

### Example 2—Manufacturing a HEMA-MAA Hydrogel Sleeve Comprising a Mesh

### The Materials

2-Hydroxyethyl methacrylate “HEMA”, Methacrylic acid “MAA”, Ethylene glycol dimethacrylate “EGDMA”, 2,2′-Azobis(2-methylpropionamidine) dihydrochloride “Azobis” by Sigma-Aldrich UK and deionized water “DI water”. A nylon (warp knitted) mesh.

### Equipment

Weighing scale, weighing paper, spatula, a beaker made from borosilicate glass and plastic screw lid, magnetic stirring bar, magnetic stirrer, plastic petri dish, ultrasonic bath, nitrogen gas, chemical fume hood, UV curing machine (12 W LED UV at 365 nm).

An example of the constituent amounts is listed in Table 2. The total amount can be adjusted depending on the sizes of the moulded product.

TABLE 2
Calculated weights for ingredients for HEMA-MAA
gel, to be scaled as desired.

| HEMA-MAA(95-5) | Weight |
|---|---|
| HEMA | 9.5 (g) |
| MAA | 0.5 (g) |
| EGDMA | 0.23 (g) |
| DI Water | 1.14 (g) |
| Azobis | 0.11 (g) |

### Protocol

1. Place the nylon (warp knitted) mesh in a mould.
2. Measure DI water weight directly in the glass beaker. Measure all constituents and pour into the water in the beaker. Be careful not to breathe in monomer powders/MAA.
3. Place a magnetic stirrer in the beaker, place a lid on the beaker and turn the magnetic stirrer on so that it stirs at fast but stable speed at room temperature until the constituents are dissolved (transparent and clear solution), and then leave to stir for an additional 30 mins.
4. Remove oxygen from the solution by bubbling nitrogen for 30 mins and ultrasonic bath for 30 mins.
5. Pour the dissolved hydrogel solution into the mould.
6. Put the mould under UV light until the solution is cured (length of time is dependent on thickness of sample).
7. Submerge the hydrogel with the mould in an excessive volume of DI water (in this example, more than 2 L) for at least 48 hours.
8. Demould the shaped hydrogel.
9. Wearing gloves, drain water containing unreacted monomers out and rinse the HEMA-MAA samples a few more times under water.

### Example 3—Manufacturing a PAAm Hydrogel Cap Comprising a Mesh

### The Materials

Acrylamide, N,N′-Methylenebis(acrylamide) “Bis”, 2,2′-Azobis(2-methylpropionamidine) dihydrochloride “Azobis” by Sigma-Aldrich UK and deionized water “DI water”.

### Equipment

Weighing scale, weighing paper, spatula, a beaker made from borosilicate glass and plastic screw lid, magnetic stirring bar, magnetic stirrer, plastic petri dish, ultrasonic bath, nitrogen gas, chemical fume hood, UV curing machine (12 W LED UV at 365 nm). A nylon (warp knitted) mesh.

An example of the constituent amounts is listed in Table 3. The total amount can be adjusted depending on the sizes of the moulded product.

TABLE 3
Calculated weights for ingredients for PAAm gel,
to be scaled as desired.

| PAAm (12.5%) | Weight |
|---|---|
| Acrylamide | 7.1921 (g) |
| Azobis | 0.1726 (g) |
| Bis | 0.1726 (g) |
| DI water | 50 (g) |

### Protocol

1. Place the nylon (warp knitted) mesh in a mould.
2. Measure DI water weight directly in the glass beaker. Measure all constituents and pour into the water in the beaker. Be careful not to breathe in the monomer powders.
3. Place a magnetic stirrer in the beaker, place lid on the beaker and turn the magnetic stirrer on so that it stirs at a fast but stable speed and room temperature until the constituents are dissolved (transparent and clear solution), and then leave to stir for an additional 30 mins.
4. Remove oxygen from the solution by bubbling nitrogen for 30 mins and ultrasonic bath for 30 min.
5. Pour the dissolved hydrogel solution into the mould. The solution has a low viscosity so should be able to be easily poured into more complex moulds.
6. Put the mould under UV light until the solution is cured (length of time is dependent on thickness of sample).
7. Submerge the hydrogel with the mould in an excessive volume of DI water (in this example, more than 2 L) for at least 48 hours.
8. Demould the shaped hydrogel.
9. Wearing gloves, drain water containing unreacted monomers out and rinse the PAAm samples a few more times under water.

### Example 4—Method of the Disclosure

1. A method of making a device for assisting childbirth, the method comprising: moulding a hydrogel solution into a device for assisting childbirth.
2. The method according to clause 1, wherein the hydrogel solution comprises synthetic polymer fibres.
3. A method of making a device for assisting childbirth, the method comprising: moulding a hydrogel solution comprising synthetic polymer fibres into a device for assisting childbirth.
4. The method according to any one of clauses 1 to 3, wherein moulding comprises casting the hydrogel solution to create a cast comprising the hydrogel solution and synthetic polymer fibres; initiating hydrogel formation; and then curing the cast to form a device for assisting childbirth.
5. A method of assisting childbirth by a pregnant subject, the method comprising: placing a device according to the disclosure on the head of a baby within the birth canal of the subject so as to assist childbirth.
6. The method according to clause 5, wherein placing the device on the head of a baby within the birth canal comprises folding the crown or the sleeve such that there is a double layer of reinforced hydrogel between the baby's head and the birth canal.
7. The method according to claim 5 or claim 6, wherein the device is placed on the head of a baby while it is suboccipitobregmatic (well flexed) or submentobregmatic (hyperextended face presentation).
8. A method of assisting childbirth by a pregnant subject, particularly by preventing obstructed labour, the method comprising:
   using a device according to the disclosure to line at least part of a birth canal/or cover at least part of a baby in a birth canal during childbirth.

1. A device for assisting childbirth, the device comprising:
a crown for being worn on a baby's head; and
a bulbous rim defining an opening in the crown;
wherein the opening is for receiving the baby's head, and
wherein the crown and the rim are made from a hydrogel.

2. The device according to claim 1, wherein the hydrogel is reinforced with synthetic polymer fibres.

3. The device according to claim 1, wherein the hydrogel comprises water or a water-based lubricant.

4. The device according to claim 1, wherein the device is a cap.

5. The device according to claim 1, wherein the device is a sleeve comprising a first bulbous rim defining a first opening at one end, a second opening at the other end, and a second bulbous rim located between the first bulbous rim and the second opening.

6. The device according to claim 4, wherein the opening of the cap has a diameter or largest length of about 8 cm to about 14 cm.

7. The device according to claim 5, wherein the first opening of the sleeve has a diameter or largest length between about 7 cm and 12 cm.

8. The device according to claim 5, wherein the second opening of the sleeve has a diameter (or largest length) of about 16 cm.

9. The device according to claim 1, wherein the hydrogel is biocompatible.

10. The device according to claim 1, wherein the hydrogel is made from one or more polymers selected from the group consisting of: PVA, PEG, PAAm, a HEMA copolymer and PDMS.

11. The device according to claim 1, wherein the hydrogel is made from PVA, PAAm, or a HEMA copolymer.

12. The device according to claim 2, wherein the synthetic polymer fibres are woven.

13. The device according to claim 2, wherein the synthetic polymer fibres form a mesh.

14. The device according to claim 13, wherein the mesh is an open mesh, a filter mesh, a woven mesh, or a warp knit mesh.

15. The device according to claim 13, wherein the mesh is a warp knit mesh.

16. The device according to claim 2, wherein the synthetic polymer fibres are made from a selection of one or more polymers from the group including: polyamide, a polyacrylonitrile, a polyester, a polypropylene, a polybutester, a polyurea, and a polyurethane.

17. The device according to claim 2, wherein the synthetic polymer fibres are made from a polyamide.

18. The device according to claim 17, wherein the synthetic polymer fibres are made from nylon.

19. The device according to claim 1, wherein the hydrogel of the crown is between about 0.1 mm and 2 mm in thickness or between about 0.05 mm and 0.5 mm in thickness.

20. The device according to claim 1, wherein the bulbous rim is about 5-fold thicker than the crown.
